(19) United States
(12) Patent Application Publication
(10) Pub. No.: US 2026/0240906 A1
(43) Pub. Date: Aug. 20, 2026

(54) MUSCLE MASS INCREASING AGENT AND ENDURANCE IMPROVING AGENT

(71) Applicant: KOHAKU BIO TECHNOLOGY CO., LTD., Morioka-shi, Iwate (JP)

(72) Inventors: Kazuichi Sakamoto, Tsukuba-shi, Ibaraki (JP); Ben Othman Mahmoud, Tsukuba-shi, Ibaraki (JP); Kazuma Okazaki, Morioka-shi, Iwate (JP); Marie Sekita, Morioka-shi, Iwate (JP); Reiko Takeda, Morioka-shi, Iwate (JP)

(21) Appl. No.: 19/161,540

(22) PCT Filed: Feb. 29, 2024
(86) PCT No.: PCT/JP2024/007587

§ 371 Date: Sep. 2, 2025

(30) Foreign Application Priority Data
Mar. 3, 2023 (JP) 2023-032773

(51) Int. Cl. A61K 35/10; A61P 21/00

(52) U.S. Cl. CPC A61K 35/10; A61P 21/00

(57) ABSTRACT

An amber extract has been found to have a motor function improving effect, a muscle mass increasing effect, a muscle strength reinforcing effect, an energy production promoting effect, an endurance improving effect, and an anti-fatigue effect. An agent, specifically, a food or drink, a supplement, a pharmaceutical composition, or the like, comprising the amber extract as an active ingredient can produce these effects. The agent comprising the amber extract as an active ingredient can provide a composition that can increase or reinforce a muscle mass and muscle strength, further improve a motor function and endurance, and prevent fatigue.

### TECHNICAL FIELD

The present invention relates to a muscle mass increasing agent, a muscle strength reinforcing agent, an energy production promoting agent, a motor function improving agent, an endurance improving agent, and an anti-fatigue agent for exercise in a broad sense.

### BACKGROUND ART

Increase in muscle mass and improvement in muscle strength lead to improvement in motor function. Hence, for not only athletes but people who enjoy sports or training, it is also preferred to improve a motor function by increasing a muscle mass and reinforcing muscle strength. Prevention or improvement of obesity or metabolic syndrome has drawn huge interest owing to growing health consciousness, and the increase in muscle mass is considered desirable because it leads to metabolic improvement and decreasing obesity or the like.

Frailty, which is a state of physical and mental decline and is an intermediate status between being healthy and being in need of care, is of concern as the population ages. It is known that signs, such as reduction in physical activity, muscle strength, and body weight, observed in frailty are associated with each other and easily brought to a negative cycle called frailty cycle. Specifically, this negative cycle occurs such that decreased muscle strength or muscle mass ascribable to aging or disorder decreases physical activity and results in decrease in energy consumption and decreased appetite, which cause malnutrition, thereby decreasing a body weight and further decreasing the muscle strength or the muscle mass. Sarcopenia, an age-related loss of a muscle mass or muscle strength, is partly responsible for frailty. Maintenance of a muscle mass or muscle strength as well as maintenance of physical functions is also important for preventing sarcopenia and frailty.

Improvement in endurance or resistance to fatigue is important both for young people and for elderly people. Although ATP for muscle movement is reported to be required for maintaining ability to exercise for a long time, a very small amount of ATP is stored in muscle fibers. Therefore, rapid supply of ATP is necessary.

In any case, no one questions the importance of diet and exercise. However, persons who are in the state of sarcopenia have the difficulty in doing proper exercise. Even young people often have the difficulty in attaining all necessary dietary intakes. Accordingly, there is a demand for a material that can be incorporated into foods or drinks and can increase or reinforce a muscle mass or muscle strength by daily ingestion.

Various health foods, functional foods, and the like have been launched or proposed in order to achieve improvement in muscle mass or muscle strength. For example, a muscle strength maintaining agent containing a branched chain amino acid (Patent Literature 1) and a muscle reinforcing agent having a plant extract as an active ingredient (Patent Literature 2) have been proposed. Moreover, an agent that achieves improvement in endurance or resistance to fatigue has been proposed (Patent Literature 3).

However, most of the conventional health foods are insufficiently effective. No drug or supplementary food that is safe and highly effective for increasing or reinforcing or maintaining a muscle mass or muscle strength, improving endurance, or preventing fatigue has been distributed. Accordingly, there is a demand for a composition that can effectively increase or reinforce a muscle mass and muscle strength, further improve a motor function and endurance, and prevent fatigue.

Amber is a fossil formed mainly from a resin of the genus Pinus buried underground for a long period and congealed, and mainly contains the resin, essential oil, succinic acid, and the like. Amber is used in diverse manners in such a way that not only is amber used as decorative crafts or jewels as it is but amber is made into insulating materials or its shavings are used in incenses.

Attempts have been made in recent years to utilize components contained in amber in drugs for external use or oral ingestion. There are reports on, for example, techniques of blending an amber extract into cosmetics or topical skin preparations (Patent Literatures 4 and 5) or techniques of utilizing useful components in an amber extract by separating a hyaluronic acid production promoter or an angiogenesis promoter from the extract (Patent Literatures 6 and 7). A technique of utilizing a suppressive effect on the absorption of glucose (Patent Literature 8) has also been reported recently.

### CITATION LIST

### Patent Literature

Patent Literature 1: Japanese Patent Laid-Open No. 2000-026290
Patent Literature 2: Japanese U.S. Pat. No. 6,817,950
Patent Literature 3: Japanese Patent Laid-Open No. 2022-021280
Patent Literature 4: Japanese Patent Laid-Open No. 2007-314522
Patent Literature 5: Japanese Patent Laid-Open No. H9-227334
Patent Literature 6: Japanese Patent Laid-Open No. 2008-266260
Patent Literature 7: Japanese Patent Laid-Open No. 2011-256164
Patent Literature 8: Japanese Patent Laid-Open No. 2019-163343

### Non Patent Literature

Non Patent Literature 1: Yuan, Y. et al., 2017, Mol. Med. Rep., Vol. 16, pp. 7361-7366
Non Patent Literature 2: Wan, T., et al., 2019, EMBO Reports, 20: e47892,
https://doi.org/10.15252/embr.201947892

### SUMMARY OF INVENTION

### Technical Problem

Amber is beginning to be exploited in various fields. However, much remains unknown about its active ingredient, and amber is far from sufficiently utilized or exploited. The present inventors have attempted to explore a new possibility of a component contained in an amber extract, leading to its utilization or exploitation. An object of the present invention is to provide a composition that can effectively increase or reinforce a muscle mass and muscle strength and further improve a motor function. Particularly, an object of the present invention is to provide a muscle mass increasing agent, a muscle strength reinforcing agent, a motor function improving agent, an energy production promoting agent, an endurance improving agent, and an anti-fatigue agent that are incorporated as a food or drink conveniently ingestible on a daily basis.

### Solution to Problem

The present inventors have administered an amber extract to mice and as a result found that the amber extract increases and reinforces muscles, improves endurance, has an anti-fatigue effect, and improves a motor function. The present inventors have also found, from experiments using cultured cells, that the amber extract promotes ATP production, i.e., energy production, thereby completing the present invention. The present invention relates to a motor function improving agent, a muscle mass increasing agent, a muscle strength reinforcing agent, an energy production promoting agent, an endurance improving agent, and an anti-fatigue agent, comprising an amber extract as an active ingredient.

### BRIEF DESCRIPTION OF DRAWINGS

FIG. 1 is a diagram showing results of analyzing an effect of an amber extract on ability to produce ATP in myotube cells.

FIG. 2 is a diagram showing results of analyzing effects of the amber extract and succinic acid on mitochondrial activation.

FIG. 3 is a diagram showing results of analyzing effects of the amber extract and succinic acid on mitochondrial membrane potential.

FIG. 4 is a diagram schematically showing an experimental schedule of in vivo analysis using mice.

FIG. 5A is a diagram showing an effect of the amber extract on a running distance on a weekly basis.

FIG. 5B is a diagram showing an effect of the amber extract on time-dependent change in running distance of each group.

FIG. 6A is a diagram showing an effect of the amber extract on grip strength on a weekly basis. FIG. 6B is a diagram showing an effect of the amber extract on time-dependent change in grip strength of each group.

FIG. 7A is a diagram showing an effect of the amber extract on a forced swim test on a weekly basis.

FIG. 7B is a diagram showing an effect of the amber extract on time-dependent change in forced swim test of each group.

FIG. 8A is a diagram showing an effect of the amber extract on a muscle mass 4 weeks after the start of a test.

FIG. 8B is a diagram showing an effect of the amber extract on a muscle mass 5 weeks after the start of the test.

FIG. 8C is a diagram showing results of analyzing a body weight 4 weeks after the start of the test.

FIG. 9 is a diagram showing an effect of the amber extract on an ATP level in muscles.

FIG. 10 is a diagram showing an effect of the amber extract on glycogen in muscles.

FIG. 11 is a diagram showing an effect of the amber extract on lactic acid in serum.

### DESCRIPTION OF EMBODIMENTS

The present inventors have explored the possibility of an amber extract and consequently found in animal models that the amber extract increases a muscle mass and reinforces muscle strength. Thus, the amber extract functions as an active ingredient in a muscle mass increasing agent and a muscle strength reinforcing agent. The present inventors have further conducted analysis and consequently found that the amber extract improves a motor function and improves endurance. From results of experiments using animal models or cells, the present inventors have found that the amber extract promotes energy production and thereby improves endurance and has an anti-fatigue effect, thereby completing the present invention. Specifically, the amber extract not only increases a muscle mass and reinforces muscle strength but is effective for promoting energy production, and as such, improves endurance and a motor function for exercise in a broad sense including daily activities or labor, and exerts an anti-fatigue effect. Thus, the amber extract is effective not only as a muscle mass increasing agent and a muscle strength reinforcing agent but as a motor function improving agent, an energy production promoting agent, an endurance improving agent, and an anti-fatigue agent.

### Form for Use as Food or Medicament

The muscle mass increasing agent, etc. of the present invention is particularly useful as a food or drink conveniently ingestible on a daily basis. Thus, the following muscle mass increasing agent, muscle strength reinforcing agent, motor function improving agent, energy production promoting agent, endurance improving agent, and anti-fatigue agent can be used as an active ingredient in a functional food, a health food, and the like.

Examples of the form of the muscle mass increasing agent, etc. of the present invention for use as a food include drinks such as fruit juice drinks, tea drinks, coffee drinks, refreshing drinks, dairy drinks, carbonated drinks, alcoholic drinks, energy drinks, sport drinks, and concentrates and prepared powders of these drinks. Examples of the food include bread, cake, noodles, confectionery, jellies, frozen foods, ice creams, dairy products, and various foods. Moreover, the muscle mass increasing agent, etc. of the present invention can be provided in a form such as a tablet, a capsule, or a syrup as a supplement or the like. The muscle mass increasing agent, etc. of the present invention may be provided as a pharmaceutical composition. The pharmaceutical composition is preferably administered orally and preferably formulated into a dosage form for oral use. Examples of the dosage form for oral use include tablets, capsules, elixirs, and microcapsules, to which a proper excipient can be added. The muscle mass increasing agent, etc. of the present invention, when provided in these forms, can be supplemented with a compound or an extract that complements or reinforces the action thereof, for example, an amino acid such as leucine, royal jelly, or a clove extract.

### Method for Producing Amber Extract

In Examples given below, extraction was performed using an organic solvent. However, the amber extract may be produced by use of any method such as a supercritical extraction method, a hot water extraction method, or an enzymatic reaction extraction method. In this context, the amber extract described in the present invention refers to amber itself, a processed product of amber obtained by processing such as grinding or shredding, or an extract obtained by extraction from amber or its processed product with a solvent or by a supercritical extraction method or any of other methods. The amber extract also includes fractions of the extract from which the solvent or insoluble matter has been removed, their purified products, and the like.

For extraction with an organic solvent, examples of the solvent can include water, alcohols such as methanol, ethanol, 1,3-butanediol, propylene glycol, and glycerin, esters such as ethyl acetate and methyl formate, nitriles such as acetonitrile, ethers such as diethyl ether and tetrahydrofuran, halogenated hydrocarbons such as chloroform and methylene chloride, and ketones such as acetone and methyl ethyl ketone. One or two or more of these solvents may be used singly or as a mixture. Among them, water or an alcohol is preferred, and a hydrous alcohol is more preferred. The alcohol is preferably ethanol. The water content is preferably 1% by mass to 60% by mass, more preferably 10% by mass to 50% by mass. A water content higher or lower than this range may deteriorate extraction efficiency.

For an extraction method, for example, a solvent is added in 2 to 20 times the amount of ground amber, which can then be dipped in the solvent at room temperature for several days or at a temperature near the boiling point for several hours. Then, insoluble matter is removed by filtration or the like, and the resultant can be, for example, concentrated under reduced pressure. The resulting residue may be purified by column chromatography with a column packed, for example, with silica gel, silica gel chemically modified with an octadecylsilyl group, a phenylhexyl group, or the like, a resin for size exclusion chromatography, or a resin for ion exchange chromatography.

Hereinafter, the present invention will be described with reference to Examples.

### Preparation of Amber Extract

### EXAMPLE 1

### Method for preparing amber extract—1

An amber extract was prepared by use of the following method: amber is ground into an appropriate size with a jet mill grinder or the like for easy solvent extraction. The amber powder was subjected to extraction at 40° C. for 1 hour with 50% ethanol added in 20 times the amount thereof, followed by filtration. The filtration cake was subjected again to extraction at 40° C. for 1 hour with 50% ethanol added in 20 times the amount thereof, followed by filtration. The filtrates were combined, concentrated, and dried.

### EXAMPLE 2

### Method for Preparing Amber Extract—2

The amber powder is left overnight at room temperature with 50% ethanol added in 4 times the amount thereof, followed by filtration. The filtration cake is left overnight at room temperature again with 50% ethanol added in 4 times the amount thereof, followed by filtration. The step of adding 50% ethanol and leaving the mixture overnight, followed by filtration was repeated a total of five times, and all the filtrates were combined, concentrated, and dried.

In the case of obtaining an amber extract by a supercritical method, the following method can be adopted.

### Method for Preparing Amber Extract by Supercritical Method—1

To 1 part by weight of the amber powder, 100 parts by weight of carbon dioxide in a supercritical state and 5 parts by weight of ethanol as an entrainer can be continuously supplied at a flow rate of 10 mL per minute and brought into contact with the extraction material at 80° C. at 30 MPa, followed by extraction operation for 71 minutes to obtain a supercritical extract of amber.

### Method for Preparing Amber Extract by Supercritical Method—2

To 1 part by weight of the amber powder, 100 parts by weight of carbon dioxide in a supercritical state and 5 parts by weight of 50% ethanol as an entrainer can be continuously supplied at a flow rate of 10 mL per minute and brought into contact with the extraction material at 80° C. at 30 MPa, followed by extraction operation for 68 minutes to obtain a supercritical extract of amber.

### Analysis Using Cultured Cell

The amber extract obtained through extraction by the method of Example 1 was analyzed for its action using myoblasts C2Cl2. The C2Cl2 cells are cells established from mouse regenerating muscles (striated muscles) and are considered to be derived from muscle satellite cells. The myoblasts, when cultured in a differentiation inducing medium, are fused and differentiated into myotube cells.

The C2Cl2 myoblasts were cultured in Dulbecco's modified Eagle medium (DMEM) supplemented with 10% FBS, penicillin (100 Us/ml), and streptomycin (100 mg/ml) under conditions of 37° C. and 5% CO₂. Differentiation into myotube cells was induced by medium replacement with DMEM containing 2% horse serum when the cells reached 95% confluency. The experiment was carried out using the differentiated C2Cl2 myotube after 5 to 7 days from the induction of differentiation.

### EXAMPLE 3

### Effect on Intracellular ATP Concentration

The amber extract was analyzed for its effect on ATP production. As a result of analyzing the amber extract of Example 1 by HPLC, succinic acid (8%), dehydroabietic acid (1%), abietic acid (1%), pimaric acid (0.2%), and isopimaric acid (1%) were detected as major components.

An effect of succinic acid was also analyzed because succinic acid, which accounts for the largest proportion of the amber extract, has been reported to have a promoting effect on skeletal muscle protein synthesis and an inducing effect on skeletal muscle fiber remodeling (Non Patent Literatures 1 and 2).

Intracellular ATP levels were measured using a luciferin-luciferase system. C2Cl2 cells were inoculated at 0.5×10⁴ cells/well to a 96-well plate and cultured at 37° C. for 48 hours until confluent. After induction of differentiation for 5 days, the cells were treated with the amber extract (25 or 50 μg/mL) prepared by the method of Example 1 or succinic acid (2.5 or 5 μg/mL) for 6 hours or 12 hours. The luminescence of each well was measured at 480/530 nm (excitation/luminescence) using a microplate reader (Synergy H1, BioTek Instruments).

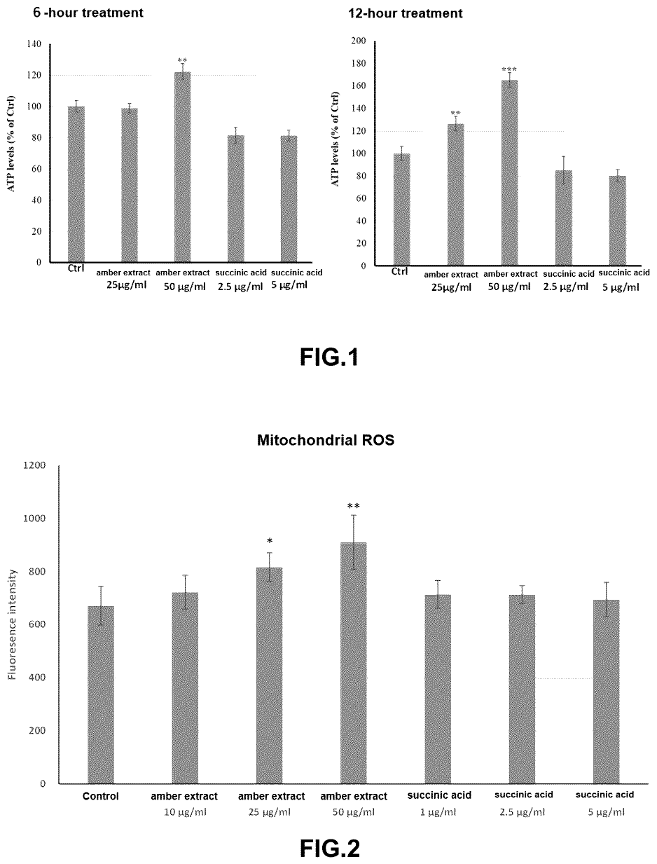

The results are shown in FIG. 1. When the C2Cl2 cells were cultured with the amber extract added at 50 μg/mL, the treatment for 6 hours was shown to significantly increase the intracellular ATP level. The treatment for 12 hours was confirmed to significantly increase the intracellular ATP, both with the 25 μg/mL amber extract and with the 50 ug/mL amber extract (in the drawing, **P<0.01, ***P<0.001; significant difference in all the drawings is indicated by *P<0.05, **P<0.01, and ***P<0.001). On the other hand, succinic acid was confirmed to cause no change in ATP concentration when used in the treatment at both the concentrations of 2.5 μg/mL and 5 ug/mL. The measured value of succinic acid contained in the amber extract was 8%, and the succinic acid concentrations of 2.5 mg/ml and 5 μg/mL substantially correspond to the concentrations of succinic acid contained in the 25 μg/mL and 50 μg/ml amber extracts, respectively.

### EXAMPLE 4

### Detection of Mitochondrial ROS

An increased intracellular ATP concentration suggests that mitochondria are activated. Accordingly, in order to confirm this, mitochondrial ROS was detected. C2Cl2 cells were inoculated at 0.5×10⁴ cells/well to a 96-well plate and cultured at 37° C. for 48 hours until confluent. After induction of differentiation for 5 days in a differentiation inducing medium, the cells were treated with the amber extract (10, 25, or 50 Mg/ML) or succinic acid (1, 2.5, or 5 μg/mL) for 24 hours. The mitochondrial ROS was measured at 485/565 nm (excitation/fluorescence) using Mitochondrial ROS Detection Assay Kit (Cayman Chemical Company).

The results are shown in FIG. 2. Significant mitochondrial activation was confirmed in the cells cultured with the amber extract added at 25 μg/mL and 50 μg/mL. On the other hand, no mitochondrial activation was confirmed in the cells supplemented with succinic acid.

### EXAMPLE 5

### Detection of Mitochondrial Membrane Potential

Mitochondrial membrane potential serving as an indicator for mitochondrial activation was also analyzed. C2Cl2 cells were inoculated at 0.5×10⁴ cells/well to a 96-well plate and cultured at 37° C. for 48 hours until confluent. After induction of differentiation for 5 days in a differentiation inducing medium, the cells were treated with the amber extract (10, 25, or 50 μg/mL) or succinic acid (1, 2.5, or 5 μg/mL) for 24 hours. The mitochondrial membrane potential was measured at green: 485/535 nm (excitation/fluorescence) and red: 535/595 nm (excitation/fluorescence) using JC-1 MitoMP Detection Kit (Dojindo Laboratories).

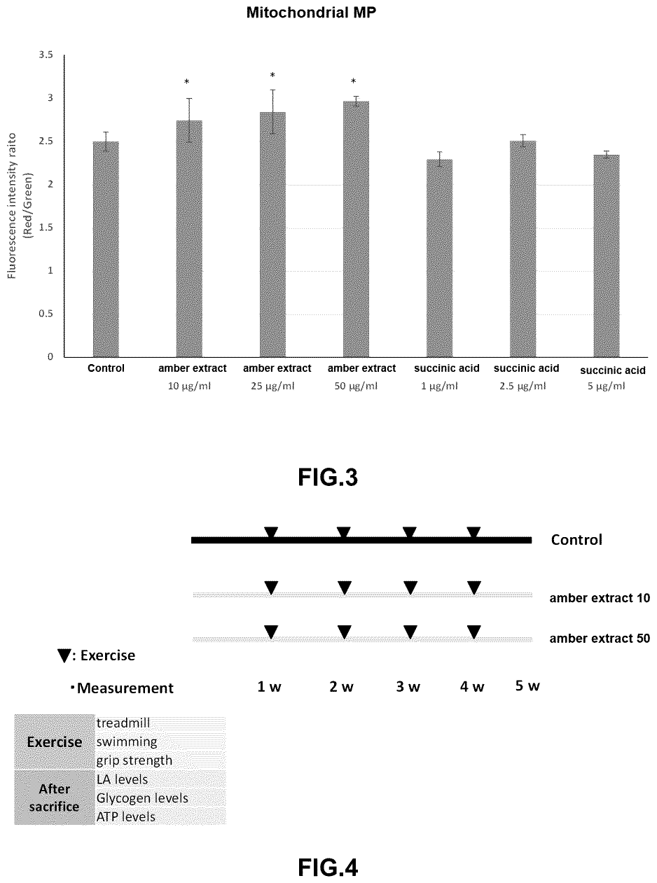

The results are shown in FIG. 3. The membrane potential indicating mitochondrial activation was significantly increased in the cells cultured with the amber extract added at the concentrations of 10 μg/ml, 25 μg/mL, and 50 μg/mL, showing that mitochondria were activated. On the other hand, no mitochondrial activation was confirmed in the cells supplemented with succinic acid.

These results demonstrated that the amber extract activates mitochondria and increases an intracellular ATP concentration. No increase in ATP concentration ascribable to mitochondrial activation was confirmed for succinic acid added at a concentration equivalent to that in the amber extract. Therefore, the amber extract is considered to contain a substance that acts strongly on mitochondrial activation. As mentioned later, succinic acid is known to have a function of increasing skeletal muscle protein, suggesting that the amber extract contains a substance that brings about a synergistic effect on the action of succinic acid.

### EXAMPLE 6

### Analysis Using Mouse

Five-week-old male C57BL/6J mice were raised for acclimatization for 1 week and then randomly divided into three groups given below each involving 12 mice. All the groups were subjected to a running test with a treadmill, a forced swim test, and grip strength measurement every week to confirm ability to exercise (FIG. 4).
Group 1: control
Group 2: oral administration of the amber extract at 10 mg/kg 5 days a week
Group 3: oral administration of the amber extract at 50 mg/kg 5 days a week

The amber extract was dissolved so as to attain a final ethanol concentration of 5% or less. The solution was orally administered five times a week using an injector such that the dose was 10 mg or 50 mg per kg of mouse body weight by the administration of 200 to 300 μL of the whole solution. Change in ability to exercise is shown below.

### (1) Effect of Amber Extract on Running Distance

One day before the first trial of exercise, each mouse was placed on a treadmill for 15 minutes and familiarized with the running environment to start the treadmill running test. The treadmill enabled mouse's running speed to be adjusted by the speed adjustment of a rolling belt, and three exercise loads given below were applied to the mouse. A running time up to complete exhaustion was recorded as to each mouse. An average value of all the mice in each group was counted.
I: 10 m/min, 15 min
II: 15 m/min, 15 min
III: 20 m/min, run to exhaustion.

An energized grid is located at the back of the rolling belt. Each mouse continues to run because electrically shocked upon contact with the grid. However, the mouse, when completely exhausted, can no longer run even by receiving electric shock. The point in time when the mouse can no longer continue to run by contact with the back grid four or five times in 1 minute was regarded as the end of the running time corresponding to complete exhaustion.

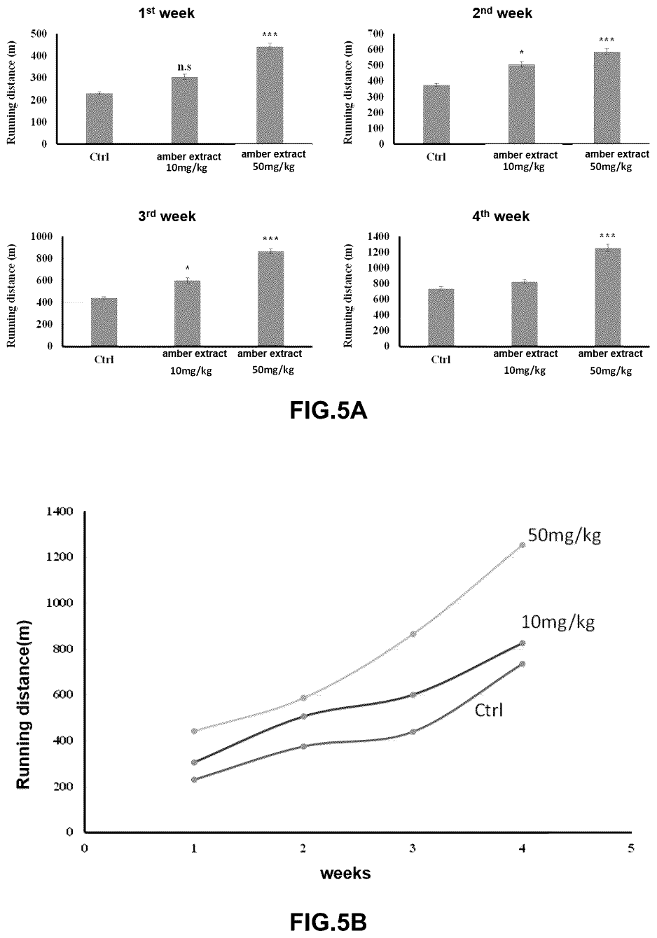

The measurement of a running distance on the treadmill was started 1 week after the start of administration of the amber extract. FIG. 5A shows the comparison of the running distance among the groups on a weekly basis. The running distance is indicated by the sum of all distances in which the mice run at the speeds I to III described above. The group given 50 mg of the amber extract per kg body weight was able to run a significantly long distance compared to the control throughout the whole test period from 1 week after the start of administration. The group given 10 mg of the amber extract per kg body weight was confirmed to have a significantly prolonged running distance on the second and third weeks after the start of administration and also confirmed to tend to increase the running distance on the other weeks. When an average running distance of each group was compared over time, both the groups given the amber extract, i.e., the groups given 10 mg and 50 mg per kg body weight, run a long distance (FIG. 5B). These results demonstrated that the amber extract confers improvement in endurance and resistance to fatigue.

### (2) Effect of Amber Extract on Muscle Strength

After running, each mouse was allowed to rest for 3 minutes in a cage, and its muscle strength was then measured using a myodynamometer (GPM-101V, Melquest Ltd.). The mouse was placed on a mesh for measurement and allowed to grasp the mesh, and the grip strength of the mouse was measured by pulling the mouse by hand until its legs got off of the mesh for measurement. Three measurements were performed for each mouse, and an average value was determined.

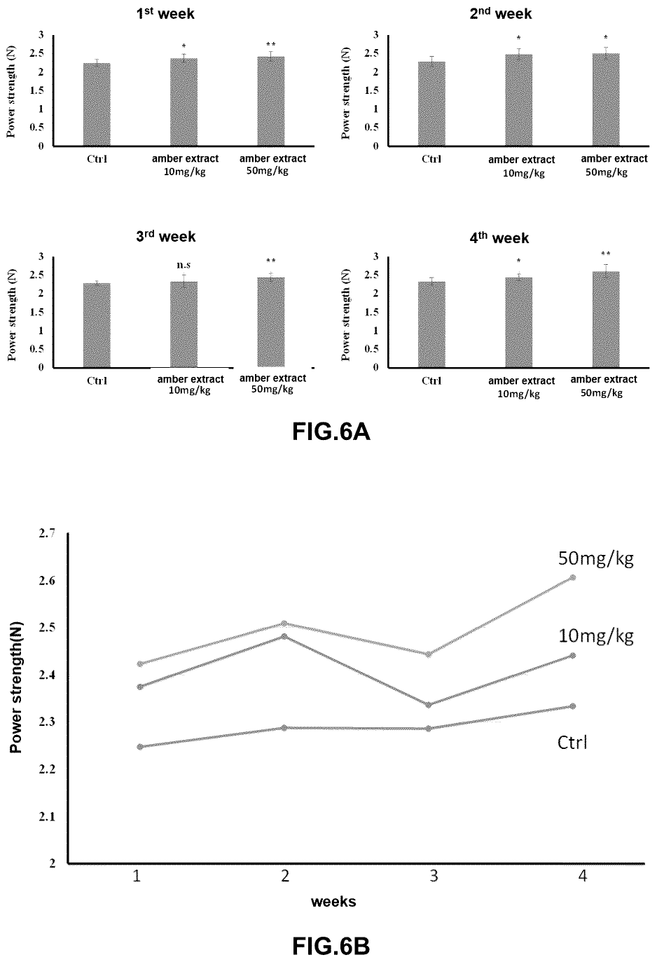

The grip strength measurement was started 1 week after the start of administration of the amber extract. FIG. 6A shows the comparison of the grip strength among the groups on a weekly basis. Both the groups given the amber extract at the doses of 10 mg and 50 mg per kg body weight were confirmed to have significantly reinforced grip strength compared to the control from 1 week after the start of administration. The group given 10 mg of the amber extract per kg body weight was confirmed to have significant difference from the control on the weeks other than the third week after the start of administration, though there was no significant difference from the control only on the third week.

Thus, the administration of the amber extract was confirmed to reinforce grip strength. When the grip strength of each group was compared over time, both the groups given the amber extract, i.e., the groups given 10 mg and 50 mg per kg body weight, had elevated grip strength (FIG. 6B). These results demonstrated that the amber extract confers reinforcement of muscle strength.

### (3) Effect of Amber Extract on Forced Swim

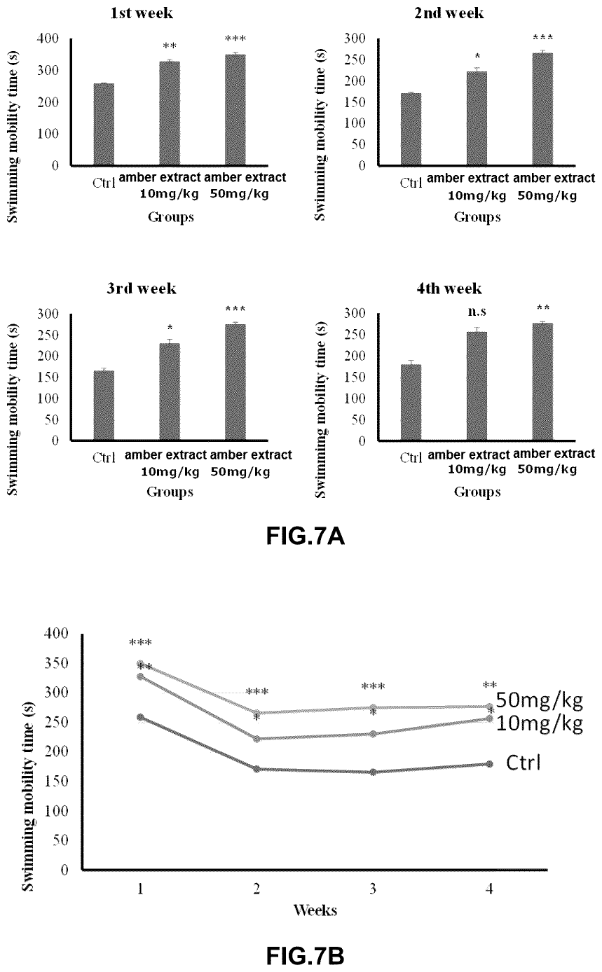

Forced swim exercise was performed in a glass beaker (diameter: 17 cm, circumference: 22 cm, height: 50 cm) in which water of 27+1° C. was put to a depth of 15 cm at which mice were not able to touch the bottom with their hind paws. One day before the first trial of the forced swim test, each mouse was allowed to swim for 5 minutes and familiarized with the swimming environment. Each mouse was individually dropped to the beaker and forced to swim for 15 minutes. The mouse swam in the beaker and additionally took actions such as climbing on the inner wall in order to get out of the beaker or immobility. A time other than an immobility time for last 10 minutes in 15 minutes was recorded as a swimming mobility time. The forced swim test was conducted four times from 1 week after the start of administration of the amber extract to the fourth week (see FIG. 4), and results were recorded (FIG. 7).

A significantly increased exercise time (swimming/climbing time) was confirmed even in the 10 mg administration group from the first week from the start of administration of the amber extract. The amber extract administration groups were confirmed to have reinforced ability to exercise, as in the running distance (FIG. 7A). Referring to time-dependent change, the exercise time on the first week is highest, and a constant exercise time is then maintained (FIG. 7B). This is presumably because the mice got used to water and acquired swimming power. The amber extract administration groups compared to the control group had a significantly long exercise time on all the weeks, showing reinforcement of ability to exercise.

### (4) Effect of Amber Extract on Muscle Weight

Four weeks after administration of the amber extract, six mice from each group were allowed to do each exercise and sacrificed after measurement of running, grip strength, and ability to swimming exercise. Soleus muscle and gastrocnemius muscle were collected from the right leg of each mouse, and their weights were measured. The remaining six mice from each group continued to undergo administration of the amber extract and were sacrificed without exercise on the fifth week, and muscle weights were measured in the same manner as above. An average value of each group was determined.

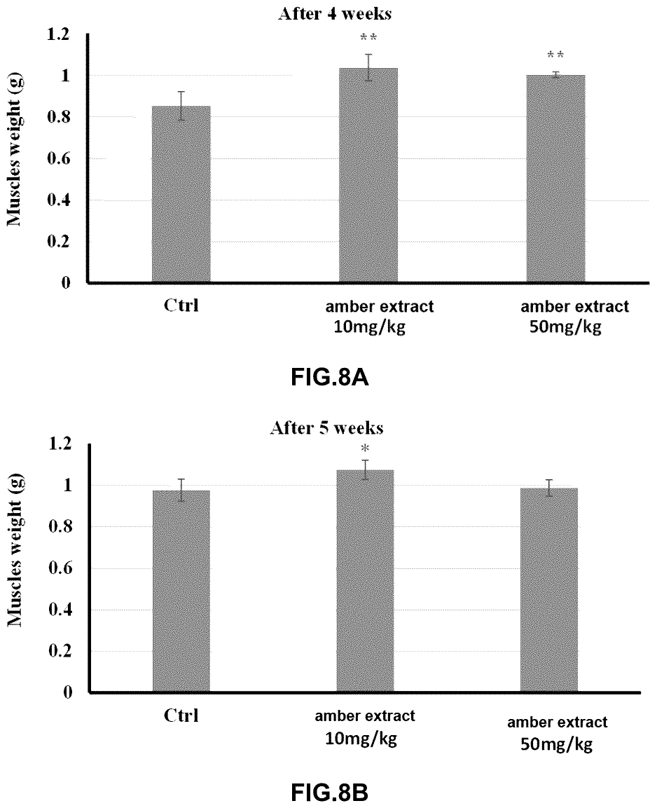

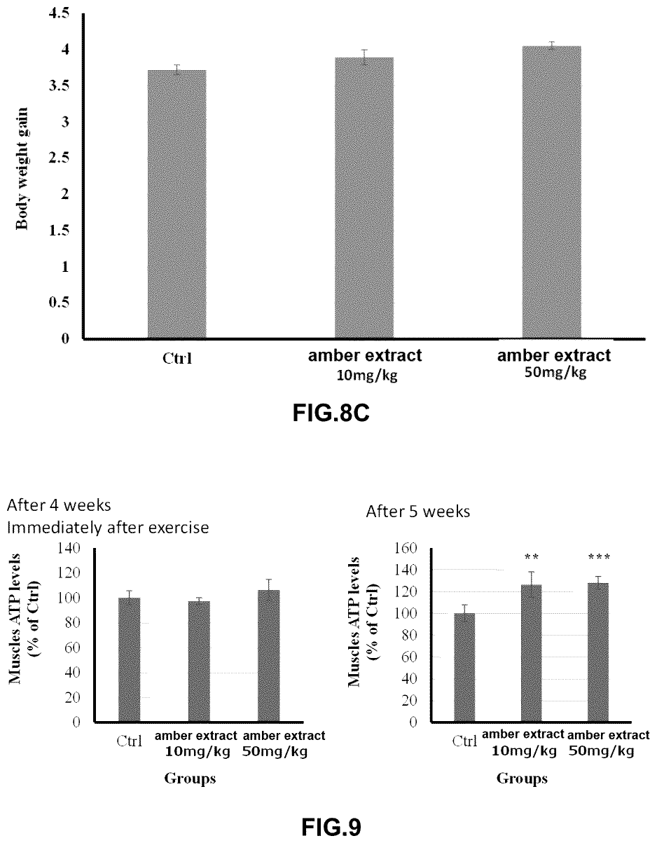

On the fourth week from the start of administration, the administered amber extract was confirmed to significantly increase the muscle mass compared to the control by both the doses of 10 mg and 50 mg per kg body weight (FIG. 8A). On the fifth week from the start of administration of the amber extract, significant increase in muscle mass compared to the control was also confirmed in the group administered 10 mg per kg body weight (FIG. 8B).

A study was made on whether this increase in muscle mass was caused by body weight gain. The mouse body weight was indicated by an average of body weights increased from the start date of oral administration (0 W) to the completion of exercise (4 W) (FIG. 8C).

Although the tendency of body weight gain was observed in the amber extract administration groups, no significant difference was confirmed in both the groups. Thus, the amber extract rarely influenced the body weight. The body weight gain in the amber extract administration groups is considered to be due to increase in muscle mass.

### (5) Effect of Amber Extract on ATP Level in Muscle

ATP concentrations in muscles (soleus muscle and gastrocnemius muscle) were measured as to each of the six mice per group sacrificed immediately after exercise four weeks after administration of the amber extract and the remaining six mice per group sacrificed on the fifth week after continuous administration of the amber extract for additional 1 week after exercise, among 12 mice allowed to do each exercise. The ATP concentrations were measured using Organs ATP measurement kit (Toyo Ink Group).

The mice sacrificed immediately after exercise exhibited no significant difference between the control group and the amber extract administration groups. By contrast, the mice sacrificed after additional 1 week were confirmed to have a significantly increased ATP level in muscles in the amber extract administration groups compared to the control group (FIG. 9). The mice sacrificed immediately after exercise on the fourth week from the start of administration of the amber extract were considered to have no difference between the groups because ATP was utilized by running or swimming. The absence of exercise for 1 week was confirmed to significantly increase the ATP level in the amber extract administration groups, showing that ability to produce ATP in muscles is increased, leading to ATP accumulation. These results are well matched with the results of the preceding experiments using cells. Increase in ATP level, which indicates promotion of energy production, is considered to result in improvement in endurance.

### (6) Effect of Amber Extract on Glycogen Level in Muscle

Glycogen levels in muscles (soleus muscle and gastrocnemius muscle) were measured in the group sacrificed after each exercise four weeks after administration of the amber extract and the group sacrificed on the fifth week after continuous administration of the amber extract in the same manner as in the preceding section (5). The glycogen contents were measured using EnzyChrom (TM) Glycogen Assay kit (BioAssay Systems, LLC).

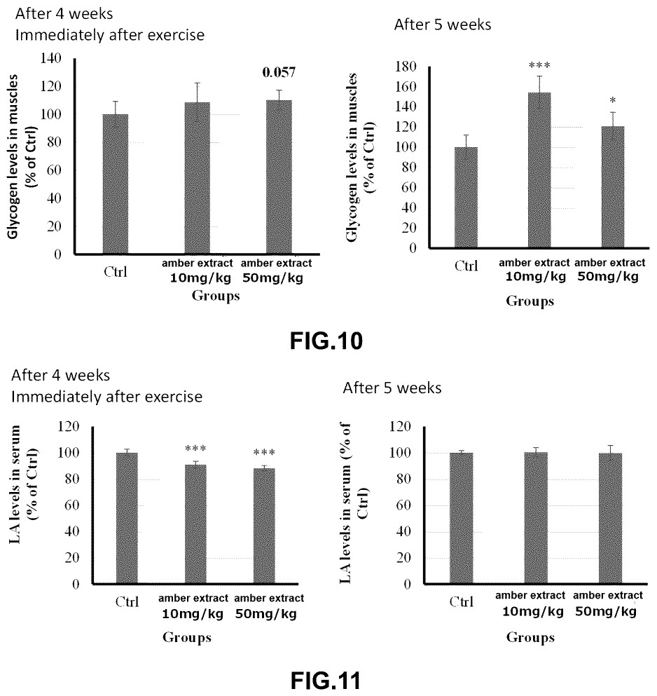

The mice sacrificed immediately after exercise exhibited no significant difference between the control group and the amber extract administration groups. The group given the 50 mg/kg amber extract was confirmed to tend to have a large glycogen content in muscles compared to the control group (p=0.057). The mice sacrificed after additional 1 week were confirmed to have a significantly increased glycogen content in muscles in the amber extract administration groups compared to the control group (FIG. 10). The mice sacrificed immediately after exercise on the fourth week from the start of administration of the amber extract were considered to have no significant difference between the groups because glycogen was utilized by running or swimming. The absence of exercise for 1 week was confirmed to increase the glycogen level in the amber extract administration groups, showing that gluconeogenesis in muscles is promoted. Increase in glycogen level in muscles is considered to lead to ATP production and result in promotion of energy production and improvement in endurance.

### (7) Effect of Amber Extract on Lactic Acid in Serum

Blood was collected from six mice per group before sacrifice allowed to do each exercise 4 weeks after administration of the amber extract and from the remaining six mice per group before sacrifice on the fifth week after continuous administration of the amber extract for additional 1 week. Lactic acid concentrations in serum were measured. Lactic acid in serum was measured using Lactate Assay kit (Cell Biolabs, Inc.).

Significant decrease in lactic acid in serum collected immediately after exercise on the fourth week from the start was confirmed in the amber extract administration groups (FIG. 11). The mice sacrificed after additional 1 week were confirmed to have no significant difference between the groups. This is because lactic acid in serum from the control group was decreased to become almost the same level as the lactic acid concentration in serum from the amber extract administration groups (results not shown). Lactic acid during the course of production is known to form hydrogen ions, which shift pH to acidity in muscles, falling into the state of muscle fatigue. Elevation in lactic acid immediately after exercise was significantly suppressed in the amber extract administration groups, showing that the amber extract has an anti-fatigue effect.

From the results described above, the amber extract is found to exert effects as a muscle mass increasing agent, a muscle strength reinforcing agent, a motor function improving agent, an energy production promoting agent, an endurance improving agent, and an anti-fatigue agent. The administration of the 10 mg/kg amber extract to mice is found significantly effective, while the administration of the 5 mg/kg amber extract is considered sufficiently effective as a result of extrapolating data. When the fact is taken into consideration that the running distance, the grip strength, and the like increase as the administration period increases, a smaller dose, for example, 2 mg/kg, can be expected to have effects when administered for a long period on the order of 3 to 4 weeks. Thus, the amber extract can be administered at 2 mg/kg or more, preferably 5 mg/kg or more, more preferably 10 mg/kg or more, per day. These values calculated on the basis of human equivalent doses correspond to 0.163 mg/kg, 0.407 mg/kg, and 0.813 mg/kg. Therefore, the amber extract can be administered to a human at 0.163 mg/kg or more, preferably 0.407 mg/kg or more, more preferably 0.813 mg/kg or more, and can be administered to a human having a body weight of 60 kg at 9.8 mg or more, preferably 24.4 mg or more, more preferably 48.8 mg more. Although there is no upper limit from a safety standpoint, the upper limit of the dose is 100 mg/kg or less, preferably 50 mg/kg or less, to a mouse and 8.13 mg/kg or less, preferably 4.07 mg/kg or less, to a human from an economic standpoint. The amber extract, if administered to a human having a body weight of 60 kg, can be administered at 488 mg or less, preferably 244 mg or less. Administration five times a week was confirmed to be sufficiently effective in the administration to the mice. Thus, ingestion at least approximately three times a week is presumed to be effective.

The amber extract can be included in various foods or effectively exploited as a supplement because, as mentioned above, the amber extract in a very small amount on the scale of mg was found to bring about many effects: it increases a muscle mass, reinforces muscle strength, improves a motor function, promotes energy production, improves endurance, and has an anti-fatigue effect. Inclusion of the amber extract in, for example, a frailty preventing agent which prevents frailty caused by reduction in muscle mass or muscle strength in elderly people, can be expected to attain increase in muscle mass, reinforcement of muscle strength, improvement in motor function, and the like. Moreover, inclusion of the amber extract in an anti-fatigue agent intended for sport devotees can be expected to have an anti-fatigue effect, as a matter of course, and can also be expected to attain improvement in endurance by promotion of energy production, and improvement in motor function as well as increase in muscle mass and reinforcement of muscle strength.

1. A motor function improving agent comprising an amber extract as an active ingredient.

2. The motor function improving agent according to claim 1, wherein the motor function improving agent is based on increase in muscle mass.

3. The motor function improving agent according to claim 1, wherein the motor function improving agent is based on reinforcement of muscle strength.

4. The motor function improving agent according to claim 1, wherein the motor function improving agent is based on promotion of energy production.

5. The motor function improving agent according to claim 1, wherein the motor function improving agent is based on improvement in endurance.

6. The motor function improving agent according to claim 1, wherein the motor function improving agent is based on an anti-fatigue effect.
